(19) United States
(12) Patent Application Publication
(10) Pub. No.: US 2020/0330721 A1
(43) Pub. Date: Oct. 22, 2020

(54) ADJUSTABLE DIFFUSER FOR MULTIPLE ESSENTIAL OILS

(71) Applicant: GREENAIR INC., WESTLAKE, OH (US)

(72) Inventor: DAVID T. CHANG, University Heights, OH (US)

(21) Appl. No.: 16/386,341

(22) Filed: Apr. 17, 2019

(51) Int. Cl. A61M 21/02

(52) U.S. Cl. CPC A61M 21/02; A61M 2021/0016

(57) ABSTRACT

A diffuser for multiple essential oils includes a housing that has at least one inlet and at least one outlet. At least two microblowers are carried by the housing to receive air though the at least one inlet. At least two bottles, each adapted to carry an essential oil, are associated with a corresponding one of the microblowers to generate a corresponding flow of oil-laden air. A controller selectively adjusts operation of each microblower to control the corresponding flows of oil-laden air to obtain a desired combination of oil-laden air exhausted through the at least one outlet.

### TECHNICAL FIELD

Generally, the present invention is directed toward diffusers. Specifically, the present invention is directed to essential oil diffusers, wherein a microblower creates particlized diffusion of the essential oil within the diffuser. More particularly, the present invention is directed to a diffuser which can adjust the amount of at least two essential oils, and which includes a fan to generate added airflow for optimal diffusion to further mix the essential oils and complete the diffusion process.

### BACKGROUND ART

It is well known that aromatherapy utilizes plant materials and aromatic plant oils which are dispersed by a diffuser. In particular, essential oils are used for the purpose of calming and relaxing those in the vicinity of the diffuser. It is also believed that aromatherapy utilizing essential oils may assist in healing.

One common method of dispersing an essential oil is to utilize aerial diffusion wherein the essential oil is dispersed by passing an airflow over the oil or by gently warming the oil. It is also known to mix the essential oil with water in a diffuser reservoir, generate a vapor of the oil-water mixture by means of an ultrasonic transducer, and then distribute the vapor with a fan. However, the mixture dilutes the effectiveness of the oil and also requires repeated refilling of the diffuser reservoir. In other words, the reservoirs eventually run out of water which automatically triggers the diffuser to stop operating and thus no oil is dispersed, thereby limiting its effectiveness. The water and oil vapor mixture also potentially introduces unwanted humidity in the air.

A further drawback is that currently known diffusers do not allow for a user to select amounts of different oils to obtain a desired combination. Namely, users can only make custom blends in a separate bottle, then add to diffusers with fixed diffusion output. Alternatively, it is known that customers opt to purchase off-the-shelf blends for diffusers. For example, a “sleep blend” provides a combination of orange oil, tangerine oil, lavender oil, chamomile oil, ylang ylang oil, and a sandalwood blend (grapeseed oil, and sandalwood oil). While such a blend may be acceptable to most users, some may dislike lavender oil, and others may detest ylang ylang oil. In any event, such blends do not allow a user to design their own blends for their own specific purposes.

Therefore, there is a need in the art for a means to effectively diffuse a combination of different oils in a fully customizable way with adjustable airflow and strength of each oil. Moreover, there is a need to provide a simple to use interface which allows selection of a percentage amount of an oil with a percentage amount of a different oil, or an interface which allows selection of a percentage mixture of two or more oils.

### SUMMARY OF THE INVENTION

In light of the foregoing, it is a first aspect of the present invention to provide an adjustable diffuser for multiple essential oils.

It is another aspect of the present invention to provide a diffuser for multiple essential oils, comprising a housing having at least one inlet and at least one outlet, at least two microblowers carried by the housing to receive air though the at least one inlet, at least two bottles, each adapted to carry an essential oil, each bottle associated with a corresponding one of the microblowers to generate a corresponding flow of oil-laden air, and a controller selectively adjusting operation of each microblower to control the corresponding flows of oil-laden air to obtain a desired combination of oil-laden air exhausted through the at least one outlet.

Still another aspect of the present invention is to provide a diffuser for multiple essential oils, comprising a housing having an inlet and an outlet, a plurality of bottles, each adapted to carry an essential oil, a plurality of microblowers maintained in the housing, the plurality of microblowers selectively controlled to selectively disperse the essential oils as oil-laden air, and a fan drawing air in through the inlet and to diffuse the air and oil-laden air through the outlet.

### BRIEF DESCRIPTION OF THE DRAWINGS

These and other features and advantages of the present invention will become better understood with regard to the following description, appended claims, and accompanying drawings wherein:

FIG. 1 is perspective view of an adjustable diffuser for multiple essential oils according to the concepts of the present invention;

FIG. 2 is an exploded perspective view of the diffuser according to the concepts of the present invention

FIG. 3 is a cross-sectional view of the diffuser according to the concepts of the present invention;

FIG. 3A is a detailed cross-sectional view of a microblower, a manifold, and an essential oil bottle as used in the diffuser according to the concepts of the present invention; and

FIG. 4 is a perspective view, in partial cross-section, of the diffuser according to the concepts of the present invention.

### BEST MODE FOR CARRYING OUT THE INVENTION

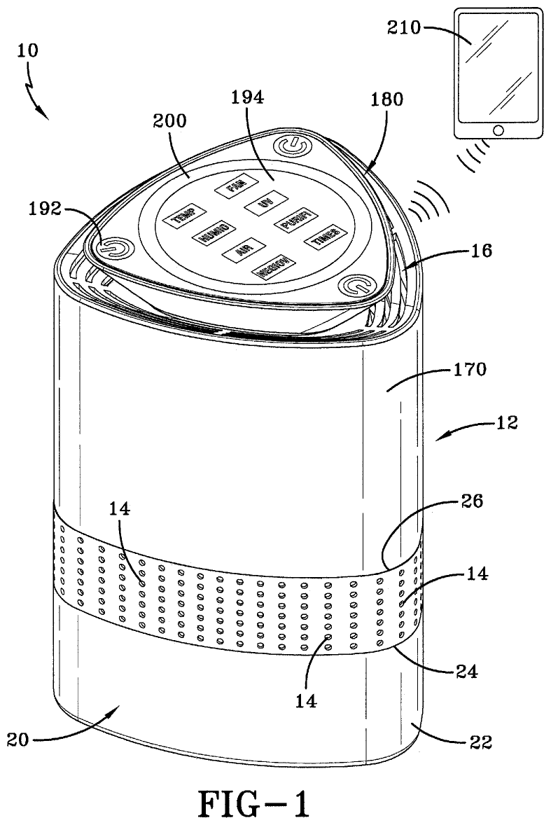

Referring now to the drawings, and in particular to FIG. 1, it can be seen that an adjustable diffuser for multiple essential oils is designated generally by the numeral 10. Generally, the diffuser 10 employs a two-stage diffusion process in combination with a feature that allows for multiple oils to be dispensed simultaneously. Moreover, the diffusion process allows for a user to selectively adjust a percentage amount of each oil to be diffused. First, each individual supply of oil is associated with a microblower which collectively generate a blended essential oil air mixture, also referred to as oil-laden air, and then a diffuser fan disperses the blended oil-laden air to ambient. It is believed that this configuration adequately blends and diffuses the essential oils in an airborne mixture instead of a mist mixture so as to enhance the benefits of the essential oils and allows for the user to provide for a unique mixing of the essential oils.

The diffuser 10 provides for a housing 12 which may be assembled from multiple components. The housing 10 provides for inlets 14 to draw in ambient air and outlets 16 which allow for diffusing of the oil-laden air in selected combinations. Although the housing 12 is shown in a substantially triangular configuration for diffusing three different types of oils, skilled artisans will appreciate that other shapes may be employed to accommodate various design elements and, in instances where any number of oils are to be blended or diffused.

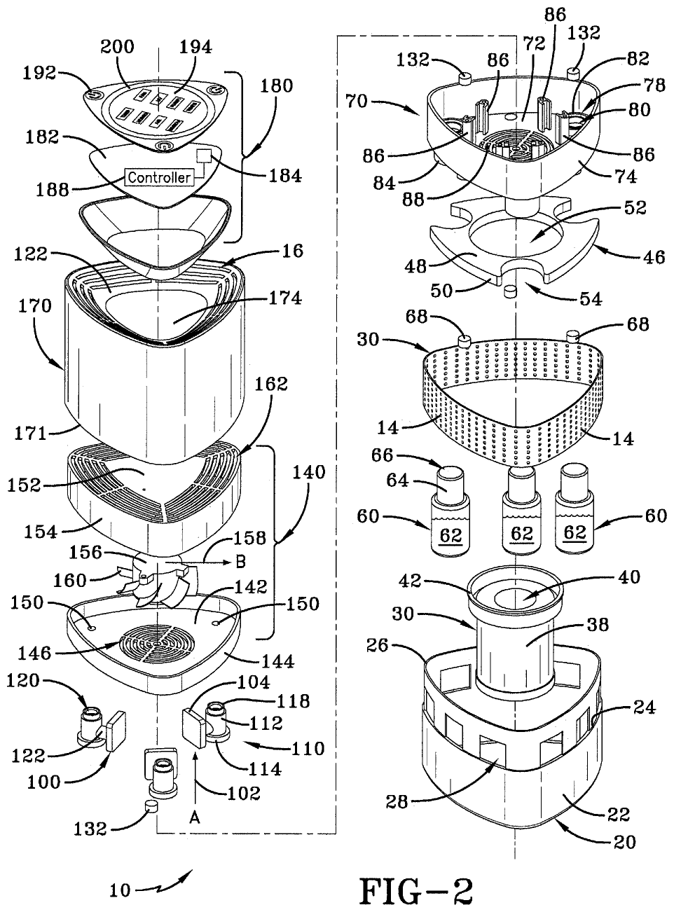

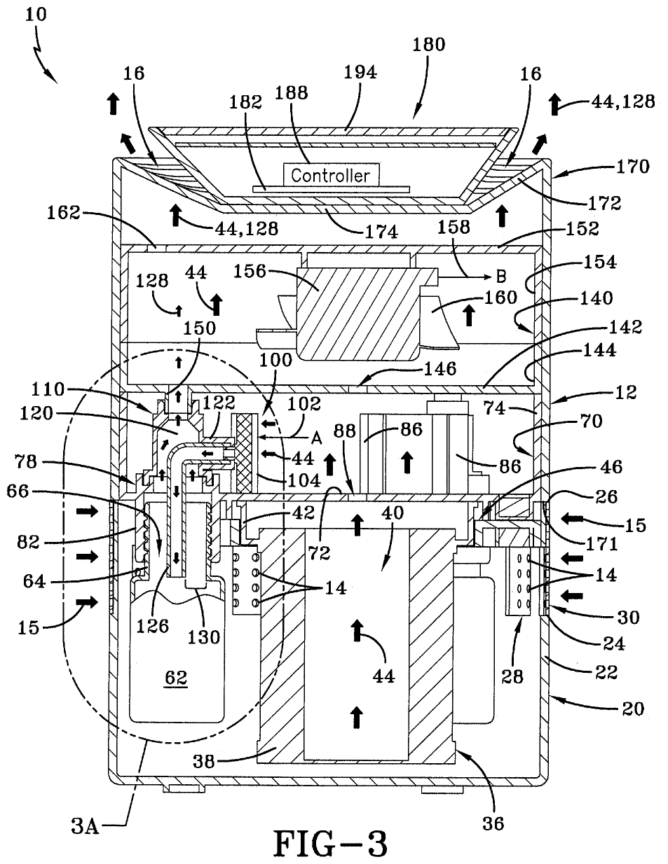

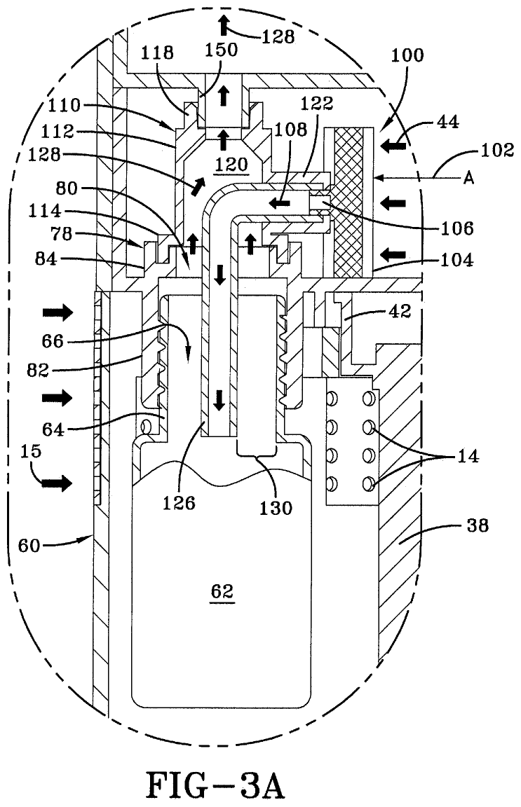

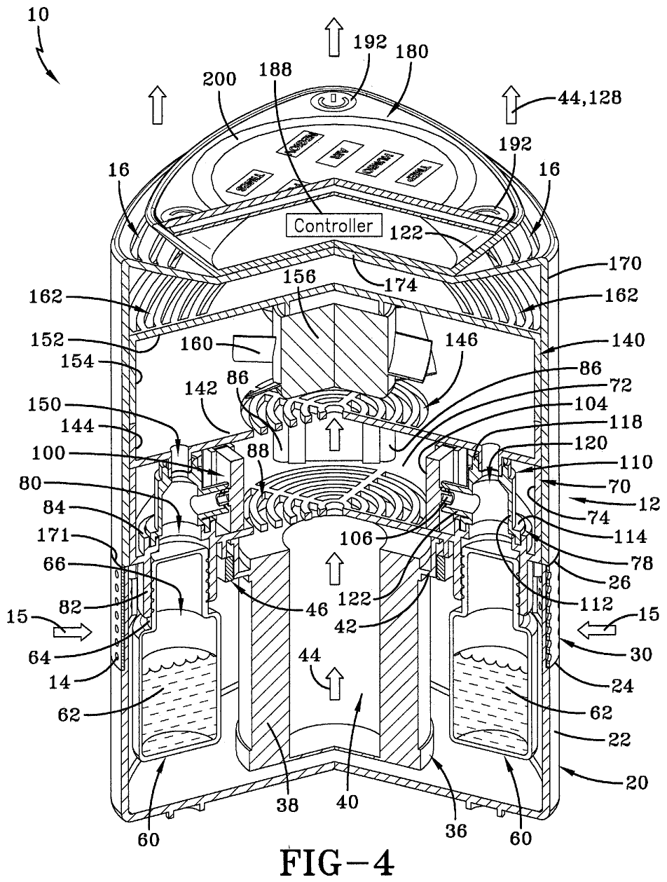

The housing 12 provides for a base 20 which includes a sidewall 22. The sidewall 22 may provide for a lip edge 24 that extends around the periphery of the base 20 and a top edge 26 which is at a top of the sidewall 24. As best seen in FIGS. 2-4, the sidewall 22 provides for a number of wall inlets 28 which may be aligned with the inlets 14. The inlets 28 are provided by a screen 30 which has a bottom edge that rests on the lip edge 24 and a top edge which is aligned with the top edge 26 of the base 20. The inlets 14 are aligned with the wall inlets 28 such that the screen 30 provides for an initial filter to prevent large objects from entering the housing 12. The screen 30 may be substantially flush with the surface of the sidewall 22, but skilled artisans will appreciate that any other configuration of screen and base may be utilized based upon design considerations.

A high-efficiency particulate air (HEPA) filter 36 may be received in the base 20. The filter 36 provides for a filter body 38 which forms an axial cavity 40, which may be closed at one end, extending substantially therethrough. Extending from and around an upper edge of the filter body 38 is an upper rim 42. Air that is drawn into the housing 12 proceeds through the inlets 14, the wall inlets 28, and then into the filter body 38. After passing through the filter body 38, the ambient air is transformed into filtered air 44 which passes axially from the axial cavity 40 into the other features of the housing 12.

A holding bracket 46 may be positioned around the filter 36 and, in particular, the upper rim 42. The bracket 46 may provide for a bracket body 48 which, in the present embodiment, has a quasi-triangle shape. Skilled artisans will appreciate that the bracket body can be configured in whatever shape is appropriate for the housing configuration. In any event, the bracket body 48 provides for a bracket periphery 50 which seals around facing surfaces of an upper inside edge of the base 20. The bracket body 48 provides for a bracket hole 52 which is aligned with the axial cavity 40 and which substantially seals around the upper rim 42. As a result ambient air drawn into the base 20 flows through the filter body 38, and into the axial cavity 40. In the present embodiment, the bracket body 48 provides for semi-circular notches 54 at each of the corners of the triangular shaped bracket body 48.

A bottle 60 is received within the base and is sized so as to fit within a respective semi-circular notch 54. This provides for further sealing of the holding bracket 46 with respect to the filter 36. In the present embodiment, three bottles 60 are shown, although skilled artisans will appreciate that two bottles, or more than three, could be employed depending on a particular end use. Each bottle 60 contains a supply of essential oil 62 wherein skilled artisans will appreciate that each bottle may carry a different essential oil as desired by the end user. Each bottle has a neck 64 which may have a reduced diameter from the bottle. Each neck 64 provides for a neck opening 66. Various sizes of bottles may be used such as 5 ml, 10 ml, 15 ml, or any other appropriate size.

A plurality of lower magnets 68 may be secured to an underside of the bracket body 48. The magnets 68 may be employed to secure other components within the housing to the holding bracket 46 as will be discussed.

A carrier 70 may be positionable upon the bracket 48. The carrier 70 provides for a plate 72 wherein an outer wall 74 extends upwardly from a peripheral edge of the plate 72. The plate 72 may provide for a collar 78 having a collar opening 80 extending therethrough. In the present embodiment, three collars 78 with corresponding collar openings 80 are shown, wherein the collar openings and the collars are alignable with the corresponding semi-circular notches 54 and the corresponding bottles 60. Each collar 78 provides for a neck fitting 82 which extends downwardly from an underside of the plate 72. In a similar manner, a manifold fitting 84 may extend upwardly from a top side of the collar 78. Positioned away from the collars 78 may be a corresponding set or pairs of fixture columns 86. In other words, one pair of fixture columns 86 will be associated with each collar and further with each bottle 60. The plate 72 may also provide for a plurality of carrier plate openings 88 which are aligned with the bracket hole 52 and further aligned with the axial cavity 40 of the filter body 38. Skilled artisans will appreciate that the carrier may also serve to provide further sealing around the bracket 46 so that any air received within the base 20 is directed into the filter 36.

A microblower 100 may be carried by the plate 72 and, in particular, a microblower 100 may be associated with each pair of fixture columns 86. In the present embodiment, each microblower, wherein the number of microblowers corresponds to the number of bottles of essential oil, is positioned between the fixture columns for each corresponding bottle. Each microblower 100 receives a blower signal 102 designated by the capital letter A. Each microblower 100 may have an inlet side 104 oriented toward the carrier plate openings 88 and an outlet port 106 on an opposite side of the inlet side. Each microblower may utilize a transducer sheet such that an applied voltage (blower signal 102) bends the sheet to change the volume of a pump chamber maintained by the blower. Accordingly, as the pump chamber volume increases, the pressure in the chamber decreases, and ambient air or other input air, in the present embodiment the filtered air, flows into the pump chamber. As the pump chamber volume decreases, the chamber pressure increases and forces the filtered air out of the pump chamber. The filtered air forced out of the microblower outlet port 106 is designated as blower air 108.

A manifold 110 may be associated with each bottle 60 and likewise with each corresponding microblower, and each collar 78. The manifold 110 may provide a substantially cylindrical wall 112 wherein a collar fitting 114 is at a bottom edge of the wall and is configured to mate and fit with the manifold fitting 84. Opposite the collar fitting 114 may be a manifold outlet 118. Extending through the manifold 110 and in particular the wall 112 is an aperture 120. Extending from the wall 112 may be a manifold inlet 122 which extends radially from the wall 112 and which has an opening therethrough which is contiguous with the aperture 120. The manifold inlet 122 may be sized to receive blower air 108 from the outlet port 106 of the microblower 100.

In some embodiments, an inlet tube 126 may be received within the manifold, and in particular one end of the tube may be associated with the manifold inlet 122 so as to be directly connected to the outlet port 106. The inlet tube 126 is routed through the aperture 120 and extends through the collar fitting 114 and the manifold fitting 84. In the present embodiment an opposite end of the inlet tube 126 extends into the neck opening 66. And the inlet tube may be positioned so as to be placed above a surface of the oil 62, but skilled artisans will appreciate that in some embodiments the inlet tube may extend into the oil a predetermined amount. In any event, the blower air 108 that is generated by the microblower may be directed through the inlet tube into the bottle 60. The inlet tube 126 has an outer diameter that is smaller than the inner diameter of the neck 64. Accordingly, the difference between the neck's inner diameter and the inlet tube's outer diameter provides for a clearance 130. Accordingly, as blower air 108 is directed into the bottle, oil-laden air 128 is generated. This oil-laden air then passes through the clearance 130 and into the manifold aperture 120. The oil-laden air 128 then passes out through the manifold outlet 118.

A plurality of upper magnets 132 may be secured to a top side of the carrier plate 72 and are aligned so as to be in relatively close proximity to the lower magnets 68 associated with the bracket body 48 when those two parts are brought together. As a result, the carrier 70 is positionable upon the bracket body 48 and detachably held in place by virtue of the magnets 68 and 132 being coupled to one another. This attachment feature allows for quick assembly and disassembly of the carrier 70 and other features of the housing with respect to the base 20. Skilled artisans will appreciate that other detachable features such as indents or other types of detachable fasteners may be employed in place of the magnets. Such a feature allows for easy replacement or refilling of the bottles 60.

A fan/motor compartment 140, which in the present embodiment comprises two pieces, is positioned above the carrier 70. The compartment 140 may provide for a compartment plate 142, which in the present embodiment is the bottom piece of the compartment, and which may rest upon the outer wall 74 of the carrier 70. The compartment plate 142 may provide for compartment sidewalls 144 which extend upwardly from an outer periphery of the plate 142. The plate 142 may also provide for a plurality of compartment plate openings 146 which may be aligned with the carrier plate openings 88 and which are aligned with the bracket hole 52 and the axial cavity 40 of the filter 36. The plate 142 may also provide for a plurality of manifold outlet ports 150 wherein the number of ports 150 correspond to the number of essential oil bottles and the other associated components. The outlet ports 150 are sized so as to fit on the manifold outlets 118 and may be configured to allow for the flow of the oil-laden air 128 from the corresponding bottles 60.

A motor plate 152, which comprises a top piece of the compartment 140, provides for downwardly extending sidewalls 154 which are supported by a top edge of the compartment sidewalls 144. A motor 156 may be carried by the motor plate 152 wherein the motor is operated by a motor signal 158 also designated by the capital letter B. When an appropriate signal 158 is received by the motor 156, the motor turns a fan 160 so as to draw air in through the base and exhaust air out the outlets 16. To accomplish this airflow, the motor plate 152 provides for motor plate openings 162 which are generally arranged around the outer periphery of the plate 152. The motor plate openings 162 are generally aligned with the manifold outlets 118 and the outlet ports 150. Skilled artisans will appreciate that the filtered air 44 and the oil-laden air 128 come together within the fan/motor compartment 140. This is accomplished by rotation of the fan 160 to draw air in through the inlets 14 wherein the generated filter air 44 is drawn into the compartment with the microblowers wherein a portion of the filtered air is used by the microblowers to force air into the bottles which generates the oil-laden air 128. The openings in the bottom of the fan/motor compartment also draw in a portion of the filtered air 44 for combination with the oil-laden air.

An outer cover 170 may be positioned onto the fan/motor compartment 140 and the carrier 70 so as to enclose these components. In the present embodiment, the fan/motor compartment is held by the cover such that the cover and the fan/motor compartment can be disassembled from the base so as to expose the carrier. Skilled artisans will then appreciate that the carrier 70 may be removed or detachably disconnected from the bracket 46 in view of the magnets 68 and 132 so as to allow for replenishment or replacement of the bottles 60. Once the bottles are replaced, the user will align the manifolds and their associated inlet tubes into the corresponding bottles, and in such a way that the magnets reengage with one another. Next, the outer cover 170 and the associated fan/motor compartment 140 are assembled onto the carrier and the base. The outer cover 170 provides for a lower edge 171 which is positioned adjacent or in contact with the top edge 26 of the base 20 when fully assembled. The outer cover 170 provides for a cover plate 172 which may be tapered inwardly in some embodiments. The cover plate 172 provides the outlets 16 about an outer periphery thereof. Accordingly, the combined and blended airflow of the filtered air 44 and the oil-laden air 128 flows through the motor plate openings 162 into the area between the cover plate 172 and the motor plate 152. This air is then released or exhausted through the outlets 16. The cover plate 172 may provide a central base 174 wherein the outlets 16 surround the base.

A control module 180 may be positioned on or otherwise secured to the central base 174. The control module 180 may carry a circuit board 182 which receives power input 184 and a controller 188. The power input 184 may receive electrical power from any source of electricity such as a USB connection, batteries, mains power of the like. The controller, which provides for the necessary hardware, software, and memory to implement operation of the diffuser, receives the signals A and B so as to control operation of the microblowers and the motor, and to also receive input from the user and the electrical power. LED lights may be associated with the controller and carried on the circuit board so as to illuminate an input panel 194 that is maintained on top of the control module 180 for access by the user. The input panel 194 may provide for a number of different inputs and displays so as to allow for the operator to control operation of the diffuser. The input panel 194 may include a plurality of on/off switches 192 and a plurality of other inputs to control various features. The input panel 194 may also provide an adjustment setting ring 200.

The control module 180 and the controller 188 may also allow for remote wireless communication with other wireless devices such as a smartphone 210 as shown in FIG. 1. A mobile application may be stored and operated on the smart phone 210 which is paired and linked so as to enable remote operation of all the features of the diffuser 10 via a Bluetooth® or other similar wireless protocol. For example, the application may allow the user to set the desired percentages of the essential oils to be blended and diffused as described herein. Other features of the diffuser such as fan speed, operational status, and essential oil levels may be monitored or controlled by the smartphone 210.

In operation, the user, after assembling the essential oil bottles into the base and assembly of the structural components as described above, will power the unit by turning on a master on/off switch. In some embodiments actuation on any one of the on/off switches 192 may provide this function. Actuation of one of the switches 192 will allow the user to select an essential oil and set a dispensing percentage which may be from zero to one hundred percent. This adjustment may be made by the user touching the ring 200 and sliding their finger along the ring until a desired percentage value is reached as evidenced by a display that is part of the input panel 194 and then removing their finger from the ring. By utilizing the adjustment setting ring 200, the user will select a percentage value from zero to one hundred percent. This process is then repeated for all of the essential oil bottles maintained in the diffuser 10. Based on the percentage settings, the controller 188 may adjust the amount of power delivered to each corresponding microblower which controls the amount of filtered blower air 108 delivered to each essential oil bottle. This in turn determines a relative amount of essential oil-laden air 128 from each essential oil bottle placed in combination with the filtered air. Once the settings are complete, the user will start operation of the device by selecting one of the inputs so as to turn the motor/fan on so as to draw in ambient air into the housing. The motor/fan may operate at various settings such as low/medium/high. Somewhat simultaneously the microblowers are energized so as to direct the filtered air into the bottle so as to generate the oil-laden air. As described above, the various oil-laden airflows and the filtered air are blended or combined and the fan exhausts the combined oil-laden air and filtered air into ambient.

In another embodiment, the adjustment of the oil-laden airflows may be set by using color codes which associate one bottle with a particular color. Next, the adjustment setting ring will be illuminated with the associated colors and the percentages will be shown so that the user can touch the adjustment ring with one or two fingers so as to selectively adjust a percentage amount each essential oil will contribute to the combined airflow. In other words, one essential oil may be set to thirty percent, a second to thirty percent, and a third to forty percent so as to obtain the desired combination of diffuser oils to be diffused. Skilled artisans will appreciate that any combination of percentages may be deployed.

Based on the foregoing the advantages of the present invention are readily apparent. The housing and associated microblowers and diffuser fan may be configured so as to allow for a user to selectively adjust the amount of essential oil to be dispersed from each essential oil bottle. This provides any number of combinations of oil so as to provide a desired, and fully customizable aromatherapy experience with waterless and undiluted essential oil diffusion using filtered air.

Thus, it can be seen that the objects of the invention have been satisfied by the structure and its method for use presented above. While in accordance with the Patent Statutes, only the best mode and preferred embodiment has been presented and described in detail, it is to be understood that the invention is not limited thereto or thereby. Accordingly, for an appreciation of the true scope and breadth of the invention, reference should be made to the following claims.

What is claimed is:

1. A diffuser for multiple essential oils, comprising:
a housing having at least one inlet and at least one outlet;
at least two microblowers carried by said housing to receive air though said at least one inlet;
at least two bottles, each adapted to carry an essential oil, each said bottle associated with a corresponding one of said microblowers to generate a corresponding flow of oil-laden air; and
a controller selectively adjusting operation of each said microblower to control the corresponding flows of oil-laden air to obtain a desired combination of oil-laden air exhausted through said at least one outlet.

2. The diffuser according to claim 1, further comprising:
a fan drawing ambient air into said housing through said at least one inlet and exhausting ambient air and said flows of oil-laden air through said at least one outlet.

3. The diffuser according to claim 2, further comprising:
a carrier maintained in said housing, said carrier having a plate with carrier plate openings therethrough, and at least two collars each with a corresponding collar opening extending therethrough, each said collar opening associated with a corresponding one of said bottles.

4. The diffuser according to claim 3, further comprising:
at least two manifolds, each said manifold associated with a corresponding one of said collar openings and a corresponding one of said bottles, each said bottle having a neck opening, each said manifold having a wall with an aperture therethrough with a collar fitting at one end which fits onto a corresponding one of said collar openings and a side inlet extending through said wall, wherein each said microblower has an outlet port which is coupled to a corresponding said side inlet.

5. The diffuser according to claim 3, further comprising:
at least two tubes, each said tube coupled between a corresponding one of said side inlets and said neck openings.

6. The diffuser according to claim 5, further comprising:
a filter carried by said housing and interposed between said at least one inlet and said carrier plate openings, said filter having an axial cavity aligned with said carrier plate openings, wherein rotation of said fan draws ambient air through said at least one inlet, through said filter and into said filter cavity to generate filtered air which is received into an inlet side of said at least two microblowers.

7. The diffuser according to claim 6, further comprising:
a fan/motor compartment for carrying said fan, said fan/motor compartment having a compartment plate with compartment plate openings extending therethrough substantially aligned with said carrier plate openings to draw in said filtered air, and at least two manifold outlet ports extending through said compartment plate and corresponding to and aligned with said manifold outlets to draw in oil-laden air from said at least two manifolds.

8. The diffuser according to claim 7, further comprising:
an outer cover coupled to said housing and enclosing said carrier and said fan/motor compartment, said outer cover having said at least one outlet to exhaust the oil-laden air and the filtered air; and
a control module carried by said outer cover and carrying said controller, said control module having a plurality of user inputs to control operation of said controller.

9. The diffuser according to claim 1, further comprising:
a control module connected to said controller, said control module receiving user inputs to separately control each said microblower to adjust an amount of oil-laden airflow emanating from each said bottle.

10. The diffuser according to claim 1, further comprising:
a control module connected to said controller, said control module receiving user input to collectively control said at least two microblowers to adjust an amount of oil-laden airflow emanating from each said bottle.

11. The diffuser according to claim 10, further comprising:
a remote wireless communication device coupled to said controller to allow remote operation of said control module.

12. A diffuser for multiple essential oils, comprising:
a housing having an inlet and an outlet;
a plurality of bottles, each adapted to carry an essential oil;
a plurality of microblowers maintained in said housing;
said plurality of microblowers selectively controlled to selectively disperse said essential oils as oil-laden air; and
a fan drawing air in through said inlet and to diffuse said air and oil-laden air through said outlet.

13. The diffuser according to claim 12, further comprising:
a plurality of manifolds, wherein each said manifold is coupled between a corresponding one of said bottles and a corresponding one of said microblowers, wherein each said microblower has an inlet side to draw in air from said fan and an outlet port to direct air into said corresponding bottle to generate the oil-laden air.

14. The diffuser according to claim 13, wherein, each said manifold has a collar fitting at one end which is coupled to a corresponding bottle, said manifold having a manifold outlet at an opposite end with an aperture extending therebetween, and wherein a manifold inlet connects to said outlet port to deliver air from said microblower into said aperture to generate the oil-laden air which flows out said manifold outlet.

15. The diffuser according to claim 14, further comprising:
an inlet tube connected to each said outlet port at one end and routed through said aperture into a corresponding one of said plurality of bottles, each said tube and said bottle having a clearance therebetween to allow the oil-laden air to flow through said aperture and out said manifold outlet.

16. The diffuser according to claim 15, further comprising:
a filter interposed between said inlet and said fan to generate filtered air which is delivered to each said microblower and which mixes with said oil-laden prior to exiting said outlet.

17. The diffuser according to claim 15, further comprising:
a controller selectively adjusting operation of each said microblower and said fan to combine said oil-laden air and said filtered air for exhausting out said outlet.

18. The diffuser according to claim 17, further comprising:
a remote wireless communication device coupled to said controller to allow remote operation of said control module.
